(19) United States
(12) Patent Application Publication
(10) Pub. No.: US 2005/0053555 A1
(43) Pub. Date: Mar. 10, 2005

(54) Appetite control compositions and methods of use

(75) Inventor: Mark A. Pederson, Kaysville, UT (US)

Correspondence Address: MANATT PHELPS AND PHILLIPS;ROBERT D. BECKER, 1001 PAGE MILL ROAD, BUILDING 2, PALO ALTO, CA, 94304, US

(73) Assignee: Crave Busters, LLC., Beverly Hills, CA (US)

(21) Appl. No.: 10/891,712

(22) Filed: Jul. 14, 2004

Related U.S. Application Data
(60) Provisional application No. 60/486,805, filed on Jul. 14, 2003.

(51) Int. Cl. A61K 9/68; A61K 35/78

(52) U.S. Cl. 424/48; 424/762; 424/757

(57) ABSTRACT

Appetite control compositions comprise Gymnema sylvestre and an expectorant. In some embodiments, the appetite control compositions contain from about 3 mg to about 50 mg of gymnemic acid (an extract of Gymnema sylvestre), from about 10 mg to about 80 mg of glycyrrhizin (an extract of Glycyrrhiza glabra), high impact flavor, and a sweetening agent in a non-traditional dosage form. A non-traditional dosage form provides for the topical application of medicaments to the tissues of the mouth and tongue, more specifically, to the sweetness taste receptors of the tongue. By delivering gymnemic acid to the sweetness receptors of the tongue, the sensation of sweetness is blocked, thereby providing appetite control.

### CROSS-REFERENCE TO RELATED APPLICATIONS

This application claims the benefit of co-pending provisional application Ser. No. 60/486,805, filed Jul. 14, 2003, the entirety of which is incorporated herein by this reference.

### FIELD ACCORDING TO THE INVENTION

The present invention relates generally to the field of appetite control and, more specifically, to appetite control compositions and methods of use.

### BACKGROUND ACCORDING TO THE INVENTION

Appetite control compositions have been in use for years. Appetite control compositions are conventionally used to assist in weight control. Typical appetite control compositions include prescription and over-the-counter drugs, e.g., amphetamines, Acutrim®, and Dexatrim®, as well as herbal remedies, e.g., Ephedra sinica, Citrus aurantium, Garcinia cambogia, and Gymnema sylvestre.

One problem with conventional appetite control compositions is that the prescription and over-the-counter drugs can be addictive. Most of these products, such as amphetamines, stimulate the central nervous system. Over-the-counter products such as Acutrim® and Dexatrim® contain phenylpropanolamine and have recently been banned from the marketplace for safety reasons. Herbal remedies based on Ephedra sinensis and herbs that contain similar alkaloids are also central nervous system stimulants and have the potential for addiction and abuse, just as the prescription and over-the-counter drugs do.

Another problem with conventional appetite control compositions is that so-called non-thermogenic herbal remedies such as Garcinia cambogia, which contains hydroxycitric acid, are both expensive and inconvenient, requiring a daily dose of nearly three grams.

Yet another problem with known appetite control compositions is that Gymnema sylvestre has a bitter taste, requiring it to be ingested in traditional dosage forms such as capsules and tablets, which limits its use to blocking sugar absorption receptors in and beyond the stomach and requires a relatively large dose of from 80 mg to 300 mg of gymnemic acid.

It can thus be appreciated that there is a need in the art for appetite control compositions and methods of use that overcome the disadvantages of previously known appetite control compositions. In particular, it would be advantageous to provide appetite control compositions that are safe, non-addictive, economical, simple, convenient, and easy to use.

### DETAILED DESCRIPTION OF THE PREFERRED EMBODIMENTS

The appetite control compositions and methods of use in accordance with the present invention depart substantially from the conventional concepts and designs discussed above and overcome many of the disadvantages of known appetite control compositions. In some embodiments, the appetite control compositions according to the invention contain from about 3 mg to about 50 mg of gymnemic acid (an extract of Gymnema sylvestre), from about 10 mg to about 80 mg of glycyrrhizin (an extract of Glycyrrhiza glabra), high impact flavor, and a sweetening agent in a non-traditional dosage form intended to assist in weight control.

Without being bound to any particular theory of action, it is currently believed that the appetite control compositions according to the invention deliver a relatively small dose of an active ingredient, gymnemic acid, directly to the sweetness taste receptors of the tongue, which blocks the sense of sweetness and thereby produces appetite control.

To attain this, some embodiments according to the invention comprise a slow-dissolving candy or other non-traditional dosage form, whose base may contain sweeteners and high impact flavors for palatability, designed to deliver about 3 mg to about 50 mg of gymnemic acid extracted from Gymnema sylvestre herb and about 10 mg to about 80 mg of glycyrrhizin extracted from Glycyrrhiza glabra root.

The appetite control compositions according to the invention are provided in palatable, non-traditional dosage forms. As used herein, the term “non-traditional dosage form” means a dosage form that provides for the topical application of medicaments to the tissues of the mouth and tongue, more specifically, to the sweetness taste receptors of the tongue. It is advantageous to use a non-traditional dosage form that encourages the user to hold it in his or her mouth for sufficient time to allow the active ingredients to contact the tissues of the mouth and tongue and, more specifically, to contact the sweetness taste receptors of the tongue. Palatability is maximized by incorporating sweeteners and high impact flavors into the base matter of the non-traditional dosage form. Examples of suitable non-traditional dosage forms include, but are not limited to, slow-dissolving hard candies or tablets, mouth sprays, toothpastes, gum drops, chocolate bars, lozenges, and pectin or gelatin desserts. The dosage form is preferably large enough not only to facilitate contact between the gymnemic acid and the sweetness taste receptors of the tongue, but it must also be large enough to incorporate about 3 mg to about 50 mg of gymnemic acid and about 10 mg to about 80 mg of glycyrrhizin.

Gymnema sylvestre is commonly called Gurmar. It is well known in the art as a “sugar blocker.” Gymnema sylvestre contains the bitter principle gymnemic acid, which is well known for its ability to bind to the sweetness taste receptors on the tongue and to sugar absorption sites in the gut. Formerly, Gymnema sylvestre and extracts standardized to deliver gymnemic acid have been incorporated into traditional dosage forms to take advantage of its ability to block the absorption of sugar from the gut. The bitter taste of its active constituent, gymnemic acid, has previously limited the use of Gymnema sylvestre and its extracts to traditional dosage forms. Such products were designed to slow or reduce sugar absorption from the gut. Typically, the dose of gymnemic acid required to provide this “sugar blocking” effect is from 80 mg to 300 mg. Because of its bitter taste, Gymnema sylvestre or its extracts are rarely incorporated into non-traditional dosage forms. Gymnemic acid is known to selectively bind to the sweetness receptors of the tongue and eliminate the sense of sweetness for 1 to 2 hours.

Glycyrrhiza glabra root is commonly known as licorice root and is well known in the art as a sweetener, expectorant, and demulcent. Glycyrrhizin is an expectorant and demulcent that increases the texture and viscosity of the saliva in the mouth. In some embodiments, licorice root, or preferably extract of licorice root, is included in the appetite control compositions according to the invention to increase the texture and viscosity of the saliva, allowing for more efficient delivery of gymnemic acid to the taste receptors of the tongue. This reduces the amount of gymnemic acid required to block the sense of sweetness in the mouth and produce appetite control. In some embodiments, about 10 mg to about 80 mg of glycyrrhizin is incorporated into the appetite control compositions according to the invention together with gymnemic acid to increase the contact time between the gymnemic acid and the sweetness receptors of the tongue, substantially reducing the quantity of gymnemic acid required to block the sense of sweetness.

Surprisingly, the present inventor has discovered that relatively small doses between about 3 mg to about 50 mg of gymnemic acid delivered in a slow-dissolving candy or other non-traditional dosage form have the ability to block the sense of sweetness for a period of between 1 to 2 hours, thus discouraging the consumption of food and controlling the appetite. The appetite control compositions according to the invention include Gymnema sylvestre herb or, preferably, an extract of Gymnema sylvestre herb designed to maximize the quantity of gymnemic acid it contains. Standard potency Gymnema sylvestre extracts are available commercially that contain from 25 to 75% gymnemic acid.

In some embodiments, the appetite control compositions according to the invention include Glycyrrhiza glabra root or, preferably, an extract of Glycyrrhiza glabra root designed to maximize the quantity of glycyrrhizin it contains. Standard potency Glycyrrhiza glabra root extracts are commercially available that contain from 20 to 50% glycyrrhizin. Glycyrrhizin is an expectorant and demulcent that increases the texture and viscosity of the saliva in the mouth, increasing the contact time between the gymnemic acid and the sweetness receptors of the tongue. The present inventor has found that from about 10 mg to about 80 mg of glycyrrhizin delivered concurrently with gymnemic acid effectively reduces the quantity of gymnemic acid required to block the sense of sweetness for 1 to 2 hours. Delivered in one of the non-traditional dosage forms described above in combination with gymnemic acid, glycyrrhizin effectively reduces the quantity of gymnemic acid required in the composition by increasing the contact time between the gymnemic acid and the sweetness receptors of the tongue.

Although the present invention has been described with reference to particular preferred embodiments, various additions, deletions and modifications that are obvious to a person skilled in the art to which the invention pertains, even if not shown or specifically described herein, are deemed to lie within the scope according to the invention as encompassed by the following claims.

1. An edible composition in a non-traditional dosage form, comprising:
an extract of Gymnema sylvestre in an amount from about 3 mg to about 50 mg; and
an expectorant.

2. The edible composition of claim 1, wherein the expectorant comprises a salivation-inducing amount of the expectorant.

3. The edible composition of claim 1, wherein the non-traditional dosage form comprises a candy, a tablet, a mouth spray, a toothpaste, a gum drop, a chocolate bar, a lozenge, a pectin dessert, or a gelatin dessert.

4. The edible composition of claim 1, wherein the non-traditional dosage form comprises a dosage form enabling time-extended topical application of the edible composition to an oral tissue.

5. The edible composition of claim 4, wherein the oral tissue comprises a sweetness receptor.

6. The edible composition of claim 1, wherein the expectorant comprises an extract of licorice root.

7. The edible composition of claim 6, wherein the extract of licorice root comprises glycyrrhizin.

8. The edible composition of claim 7, wherein the glycyrrhizin comprises from about 10 mg to about 80 mg of glycyrrhizin.

9. The edible composition of claim 1, wherein the extract of Gymnema sylvestre comprises gymnemic acid.

10. The edible composition of claim 1, further comprising a flavoring.

11. The edible composition of claim 1, further comprising a sweetener.

12. A method for reducing appetite in a subject desiring the same, comprising administering to the subject an edible composition in a non-traditional dosage form, the edible composition comprising an extract of Gymnema sylvestre in an amount from about 3 mg to about 50 mg and an expectorant.

13. A method for promoting weight loss in a subject desiring the same, comprising administering to the subject an edible composition in a non-traditional dosage form, the edible composition comprising an extract of Gymnema sylvestre in an amount from about 3 mg to about 50 mg and an expectorant.

14. A method for reducing appetite in a subject desiring the same, comprising administering to the subject an edible composition in a non-traditional dosage form, the edible composition comprising gymnemic acid in an amount from about 3 mg to about 50 mg and glycyrrhizin in an amount from about 10 mg to about 80 mg.

15. A method for promoting weight loss in a subject desiring the same, comprising administering to the subject an edible composition in a non-traditional dosage form, the edible composition comprising gymnemic acid in an amount from about 3 mg to about 50 mg and glycyrrhizin in an amount from about 10 mg to about 80 mg.
